(19) United States
(12) Patent Application Publication
(10) Pub. No.: US 2007/0207145 A1
(43) Pub. Date: Sep. 6, 2007

(54) MOUSE/HUMAN CHIMERIC ANTI-PHENCYCLIDINE ANTIBODY AND USES THEREOF

(75) Inventors: S. Michael Owens, Little Rock, AR (US); H. Marie Lacy, Little Rock, AR (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: BOARD OF TRUSTEES OF THE UNIVERSITY OF ARKANSAS, Little Rock, AR (US)

(21) Appl. No.: 11/738,789

(22) Filed: Apr. 23, 2007

Related U.S. Application Data
(62) Division of application No. 10/828,782, filed on Apr. 21, 2004.
(60) Provisional application No. 60/464,190, filed on Apr. 21, 2003.

(51) Int. Cl. A61K 39/395; C07K 16/44

(52) U.S. Cl. 424/133.1; 530/387.3

(57) ABSTRACT

The present invention provides a chimeric mouse/human antibody (ch-mAb6B5) for treatment of abuse and toxicity of the arylcyclohexylamines class of drugs (i.e., phencyclidine- or PCP-like drugs). This antibody comprises light and heavy chain PCP binding regions of mouse mAb6B5, coupled to the light and heavy chain constant regions of a human kappa IgG₂ or IgG₄ isoform. Also provided are the DNA and amino acid sequences of the chimeric light and heavy chain of this antibody. Further provided are data that demonstrate that the new chimeric antibody retains the high affinity and specificity of a previously generated mouse anti-PCP monoclonal antibody (mAb6B5) yet being minimally immunogenic since it has human immunoglobulin constant region. This new medication would allow safe and effective treatment of PCP drug overdose, decrease mortality, and reduce harmful effects due to excessive and prolonged PCP drug use.

### CROSS REFERENCE TO RELATED APPLICATION

This application is a divisional application of U.S. Ser. No. 10/828,782, filed on Apr. 21, 2004, which claims the benefit of provisional application U.S. Ser. No. 60/464,190 filed on Apr. 21, 2003, now abandoned.

This invention was produced using funds from the Federal government under grant no. R01DA07610 from the National Institutes of Health and grant no. G100059-04-01-E from the National Institute on Drug Abuse. Accordingly, the Federal government has certain rights in this invention.

### BACKGROUND OF THE INVENTION

1. Field of the Invention

The present invention relates generally to the field of monoclonal antibody technology. More specifically, the present invention provides a mouse/human chimeric anti-phencyclidine (PCP) monoclonal antibody useful for treating PCP drug abuse.

2. Description of the Related Art

Phencyclidine (PCP) was originally developed in the 1950's by Parke-Davis for use as an intravenous anesthetic. Use for humans was abandoned due to significant side effects. In addition to its anesthetic and analgesic effects, PCP can produce a dose-dependent psychosis that resembles schizophrenia with behavior described as extremely agitated, bizarre, unpredictable and paranoid. When introduced as a street drug in the 1960's, it quickly became a popular drug of abuse. Many PCP abusers are brought to emergency rooms because of PCP's psychological effects or because of overdoses. These patients are often violent or very dangerous to themselves and others. Results of long-term use of PCP include memory loss, difficulties with speech and thinking, depression and weight loss. For these reasons, PCP is considered a very dangerous drug of abuse.

PCP belongs to a class of structurally related drugs called arylcyclohexylamines, which includes TCP (1-[1-(2-thienyl)cyclohexyl]piperidine) and PCE (N-ethyl-1-phenylcyclohexylamine). The pharmacological effects of PCP and related compounds are produced through interaction with several neurotransmitter systems, ion channels and catecholamine uptake systems. These sites include the so-called PCP receptor, which is within the N-methyl-D-aspartate (NMDA) receptor complex and the dopamine transporter, which may also significantly contribute to PCP abuse and psychosis. Some of the arylcyclohexylamines appear to have effects similar to PCP, except that they are even more potent. For instance, TCP and PCE are about 1.3 and 6 times respectively more potent than PCP in drug discrimination assays and PCP receptor binding assays.

Treatment of the adverse effects of PCP is difficult for several reasons. First, PCP has a very high volume of distribution (V_{d}, 6.2 l/kg in humans) and it is cleared primarily by liver metabolism, with only a small contribution from renal excretion. Second, its major sites of action in the central nervous system are far removed from the beneficial effects of most traditional treatment methods such as dialysis. Third, there is no specific antagonist for PCP adverse effects. These pharmacokinetic and receptor-mediated characteristics of PCP make it very difficult to develop effective treatment strategies.

As an alternative therapeutic strategy, drug-specific antibodies have been used to target the drug rather than the site(s) of action. The antibody medication acts as a pharmacokinetic antagonist to neutralize the drug effects, along with producing significant changes in drug distribution, metabolism, and elimination. The changes in drug disposition resulting from high-affinity antibody binding and the subsequent reductions in brain concentrations provide the major beneficial effects. These immunological treatments are of two types: active immunization with drug-protein conjugates or passive immunization with laboratory generated antibodies (usually monoclonal).

Passive administration with drug-specific, high-affinity monoclonal antibodies could have important therapeutic advantages over active immunization. First, the pharmacological properties of a monoclonal antibody medication can be carefully selected and designed for optimal affinity and specificity. Second, the structure and function of monoclonal antibodies are consistent and uniform from batch to batch, and if human (or humanized) monoclonal antibodies are used for the treatment of human diseases the possibility of allergic type reactions is greatly reduced or prevented. Third, the dose of antibody can be precisely controlled, and patients can be offered immediate immunological protection against drug effects without waiting weeks or months for a response to an active immunization protocol.

Murine Anti-PCP Monoclonal Antibody 6B5 (mAb6B5)

An anti-PCP murine monoclonal antibody was developed, named mAb6B5, (IgG1 heavy chain, k light chain) that has high affinity for PCP (K_{D}=1.3 nM) and other arylcyclohexylamines (Hardin et al., 1998). It has 50 times greater affinity for PCP than the PCP receptor, over 300 times greater affinity than PCP binding to the dopamine transporter, and thousand fold greater affinity for PCP than serum protein binding sites. Previous studies indicate that mAb6B5 had promise as an immunotherapeutic agent for PCP and PCP-like drug abuse. In a rat model for human acute PCP overdose, the antigen-binding fragment (Fab) of mAb6B5 causes a rapid and effective redistribution of PCP out of the brain (Valentine and Owens, 1996). This redistribution also produces a rapid recovery from the behavioral toxicity produced by PCP, TCP and PCE in rats (Valentine and Owens, 1996; Hardin et al., 1998). The effectiveness of the mAb6B5 Fab as an antagonist for multiple arylcyclohexylamines is important as it demonstrates the feasibility of using an antibody-based therapy to treat the adverse effects of a whole class of drugs. However, in the studies cited above, equimolar amounts of Fab were required to inhibit the action of the drugs. Unfortunately, the amount of antibody required to produce that amount of Fab as a therapeutic agent is not economically acceptable.

Further studies with the intact mAb6B5 IgG revealed promising results. In rats, a single dose of mAb6B5 IgG protects against behavioral effects of repeated intravenous PCP challenges (Hardin et al., 2002). Furthermore, a single dose of mAb6B5 IgG as low as 0.01 molar-equivalents to the PCP body burden immediately and significantly reduces locomotor activity in rats that are continuously infused with high doses of PCP (Laurenzana et al., 2003).

Therapeutic Antibodies

Antibody-based therapeutics is a rapidly advancing medical treatment area as demonstrated by the fact that a quarter of new biological products in clinical development are antibody-based. At least ten monoclonal antibodies (mAbs) have received approval from the US Food and Drug Administration (FDA) for clinical use against a variety of diseases such as rheumatoid arthritis, Crohn's disease, cancer and allograft rejection. More than 70 monoclonal antibodies are currently in commercial trials beyond Phase I and Phase II.

However, the immunogenicity of murine antibodies in humans presents major problems for their therapeutic use. Murine antibodies have three regions (see FIG. 1) that produce immune responses in humans: 1) amino acids in the constant domains of the heavy chain (occasionally the light chain) produce an anti-isotypic response (the earliest and strongest immune response); 2) conformational arrangements of amino acids in the variable regions of the heavy and light chains produce an anti-idiotypic immune response; and 3) subtle amino acid differences in the constant domains that occur in some, but not all, members of a species (polymorphisms) produce an anti-allotypic immune response. Once an anti-murine immune response has been established, it can render the therapeutic antibody ineffective by neutralization or it can cause allergic or immune complex hypersensitivity (Clark, 2000).

At present, rodents are the most commonly used source for producing antibodies for therapeutic use. To overcome the immunogenicity problems, genetic engineering strategies have been developed to make rodent antibodies more similar to human antibodies and less immunogenic. There are three genetically engineered types of antibodies known to be safe and effective in clinical trials in humans: the mouse/human chimeric, the humanized, and the fully human antibody (Clark, 2000; Reichert, 200 1) (see FIG. 2).

To engineer a mouse/human chimeric antibody, the constant domains of the murine light and heavy chains are replaced with human constant domains. Thus, the chimeric antibody retains the murine variable regions (the antigen-binding site), but the highly immunogenic murine constant domains are eliminated. Humanization of a murine antibody is similar to producing a chimera, except less of the murine antibody DNA sequence is used. Only the murine complementarity determining regions (CDRs) of the variable regions are grafted onto the DNA framework of a human antibody. The CDRs are those sequences in the antigen-binding site that directly interact with the antigen and make up only about 5% of the antibody sequence. Lastly, the fully human antibody is produced via transgenic mice whose immunoglobulin genes have been replaced with human immunoglobulin genes. These mice, when immunized, produce human antibodies (Clark, 2000).

At present there are no effective medications available to help combat the problem of PCP and PCP-like drugs abuse (for e.g. TCP, PCE). The present invention fulfills this need in the art and provides a mouse/human chimeric anti-PCP antibody suitable for immunotherapy treatment for the abuse of PCP and PCP-like drugs.

### SUMMARY OF THE INVENTION

The present invention provides a genetically engineered chimeric anti-phencyclidine (PCP) monoclonal antibody (mAb) named ch-mAb6B5 as a safe and effective human therapy for treating medical problems associated with PCP and PCP-like drug abuse.

The hapten 5-[N-(1′-phenylcyclohexyl)amino]pentanoic acid (PCHAP) was used to generate murine mAb6B5 (IgG1 heavy chain, k light chain), which has a high affinity (K_{D}=1.3 nM) and specificity for PCP, as well as other arylcyclohexylamines like TCP and PCE (Owens et al., 1988). Sequencing and X-ray crystallography studies of the mAb6B5 antigen-binding fragment (Fab) revealed a unique protein structure in the antigen-binding site that should effectively reduce the immunogenicity of the antibody when it is used therapeutically (Lim et al., 1998). This is because a tryptophan flap that closes over PCP in the binding site blocks the opening to the antigen-binding site. This structure will help inhibit the formation of PCP-like anti-idiotypic antibodies, thus preventing the formation of potentially dangerous anti-receptor antibodies.

Preclinical studies in rats show that mAb6B5 can reverse or reduce the in vivo pharmacological effects of PCP and other potent arylcyclohexylamines such as TCP. When tested in a rat model based on human chronic PCP use, a single low dose of mAb6B5 provides long-term protection against the adverse effects of PCP and significantly improves the general health status of the animals. Additionally, experimental data and species scaling from rats to humans suggests that a single 1 gm dose of mAb6B5 IgG has the capability of reducing the toxic effects of 1.29 gm/day of PCP for 6-8 weeks (Laurenzana, et al., 2003). Thus, it is feasible and economically viable to develop mAb6B5 into an antibody-based therapy for treatment of PCP abuse. mAb6B5 was converted into a chimeric antibody that can be safely used in humans. The development of an antibody-based therapy described herein for treating drug classes, rather than just one specific drug, is an exciting possibility that could provide a prototypic model for designing immunotherapies for other classes of drugs.

To circumvent the immunogenic nature of murine antibodies in human, mAb6B5 was genetically engineered into a mouse/human chimeric that had the antigen-binding site of native mAb6B5 and the constant domains of human IgG2 heavy chain and kappa light chain. The cDNA of the variable regions of light chain (V_{L}) and heavy chain (V_{H}) of mAb6B5 were ligated into mammalian expression vectors—a light chain vector (pLC-huCₖ) containing the cDNA of the human kappa constant region (huC.ₖ) and a heavy chain vector (pHC-huCγ2) containing genes for the human IgG2 constant regions (huC_{G2}) (McLean et al., 2000). The ch-mAb6B5 was expressed by co-transfecting the light chain and heavy chain vector into a non-producing murine myeloma cell line P3×63-Ag8.653. The characterization of anti-PCP ch-mAb6B5 showed it to be part mouse and part human. Additionally, it was also observed that the affinity (K_{D}=1.6 nM) and specificity of ch-mAb6B5 were unaltered by the genetic manipulation. The molecular weight (145 kD) and other chemical properties of ch-mAb6B5 were also observed to be as expected from the deduced sequences.

However, using antibody as a therapeutic agent requires large-scale production. Therefore, it is contemplated that the engineered genes for chimeric mAb6B5 will be expressed in dihydrofolate reductase (dhfr) negative Chinese hamster ovary (CHO) cells using the dhfr amplification system, an approach widely used for large-scale manufacture of recombinant proteins. High producing clones will be identified, adapted to anchorage-independent growth, and grown in bioreactors to produce enough chimeric antibody for animal studies. It is also contemplated that this protein could be expressed in large quantities in plants (e.g. corn, tobacco, rice) without the need for mammalian expression systems. A series of pharmacokinetic and behavioral studies will be performed to directly compare native and chimeric mAb6B5 in terms of effectiveness. Behavioral testing will determine the magnitude and duration of the protective effects, whereas pharmacokinetic studies will determine the dose-and-time dependent changes in drug and antibody disposition. Duplication of methodologies and conditions will allow direct comparison of results for native murine mAb6B5 and chimeric mAb6B5. The present invention provides a critical step in the translation of basic research findings into a new treatment for PCP abuse.

In one embodiment of the present invention, there is provided a composition of chimeric mouse/human monoclonal antibody. This antibody comprises human immunoglobulin constant domains and immunoglobulin variable domains of murine antibody.

In another embodiment of the present invention, there is provided a composition of an expression vector. This expression vector comprises DNA encoding human immunoglobulin light chain constant domain and immunoglobulin variable domain of murine antibody.

In yet another embodiment of the present invention, there is provided a composition of another expression vector. This expression vector comprises DNA encoding human immunoglobulin heavy chain constant domain and immunoglobulin variable domain of murine antibody.

In still yet another embodiment of the present invention, there is provided a host cell line. This host cell line comprises a chimeric light chain expression vector and a chimeric heavy chain expression vector. The compositions of these expression vectors are as described earlier.

The invention may also be described in certain embodiments relating to a method of producing recombinant chimeric monoclonal antibody. This method comprises amplification of cDNAs of variable domains of murine monoclonal antibody. Chimeric light and heavy chain vectors comprising same composition as described earlier are constructed. The cells are co-transfected with the vectors and cultured under conditions effective for expression of the recombinant chimeric monoclonal antibody.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows three types of anti-murine immune responses produced in humans.

FIG. 2 shows strategies to reduce the immunogenicity of murine antibodies.

FIG. 3 shows the structure of mouse/human chimeric mAb6B5.

FIG. 4 shows the antigen-binding fragment (Fab) of mAb6B5 inhibits the locomotor effects induced by PCP, TCP, and PCE.

FIG. 5 shows a dose-response relationship between mAb6B5 Fab and the inhibition of PCP-induced locomotor effects. Five doses of Fab ranging from 0-1.0 mol-eq were administered to rats (n=4 per group) thirty minutes after the PCP dose (closed circles). Control groups received saline-saline (open triangle) or saline after PCP (open circle). Total distance traveled was measured and expressed as a percentage of the response to PCP without Fab treatment (i.e., 100% response).

FIG. 6 shows the total distance traveled by rats pre-treated with mAb6B5 or saline and then challenged with 0.32, 0.56, and 1.0 mg/kg PCP on days 1, 4, 7, 10, and 13. Male Sprague-Dawley rats received i.v. treatments of saline, non-specific bovine IgG (1.0 mg/kg) or mAb6B5 IgG (1.0 mg/kg) on day 1. The rats were then challenged with escalating doses of PCP (0.32, 0.56, and 1.0 mg/kg) spaced 90 minutes apart. This dosing regimen was repeated on days 4, 7, 10 and 13 (totaling 15 PCP doses)

FIG. 7 shows the effects of mAb6B5 on PCP brain concentrations in rats continuously infused with PCP. Rats were implanted with s.c. osmotic minipumps filled to deliver PCP at a rate of 18 mg/kg/day. At 24 hr after implantation of the pumps, a mol-eq dose of a mAb6B5 IgG was administered intravenously. The PCP infusion continued for up to 27 days. At selected time points after administration of the antibody, brain, serum and testis PCP concentrations were measured in groups of animals (n=3 per time point). There was a complete removal of PCP from the brain within 15 min, which persisted for the first 4 hr.

FIG. 8 shows dose-dependent effects of anti-PCP mAb6B5 on the body weight of rats continuously infused with 18 mg/kg/day PCP. Mean values for each treatment group (expressed as the percentage of day 0 weight, such that each animal served as its own control). Day 0 weight represents baseline weight determined just before the start of the experiment. Sample sizes were n=4 per group for saline-saline group and all anti-PCP mAb doses except 0.003 mol Eq (where n=3, and n=6 per group for the PC-saline controls). Values represent mean+/−S.D. Because maximum weight reduction was observed on day 4, the inset shows individual values (symbols) for each animal in the study on day 4 of the experiment. The open symbols represent groups in which one rat died. Mean values for each group are represented as dash.

FIG. 9 shows an experimental strategy for cloning VH subscript and VL subscript of mAb6B5 into expression vectors.

FIG. 10 shows nucleotide (SEQ ID No. 15) and amino acid sequence (SEQ ID No. 16) of anti-PCP chimeric mAb6B5 light chain (714 nt/237 AA).

FIGS. 11A and 11B shows nucleotide (SEQ ID No. 17) and amino acid sequence (SEQ ID No. 18) of anti-PCP chimeric mAb6B5 heavy chain (1389 nt/462 AA).

FIG. 12 shows immuno-slot blot analysis of purified anti-PCP mAb6B5 and ch-mAb6B5.

FIG. 13 shows Inhibition ELISA to determine the IC₅₀ values of mAb6B5 and ch-mAb6B5.

FIG. 14 shows Inhibition ELISA to determine the specificity ch-mAb6B5.

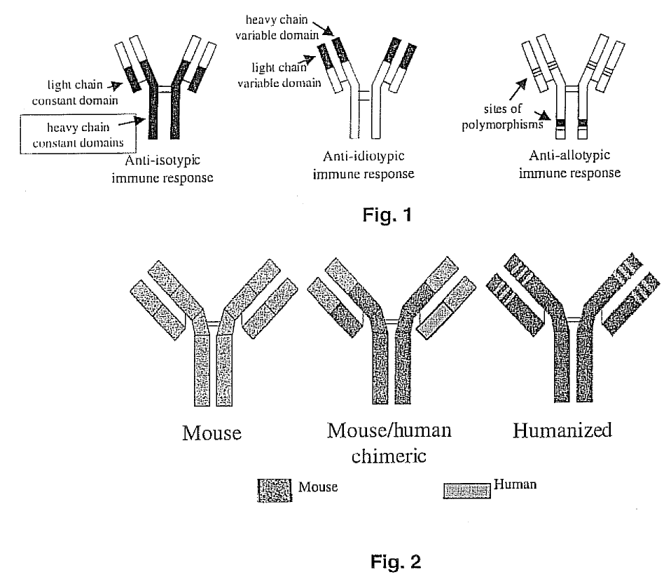

### DETAILED DESCRIPTION OF THE INVENTION

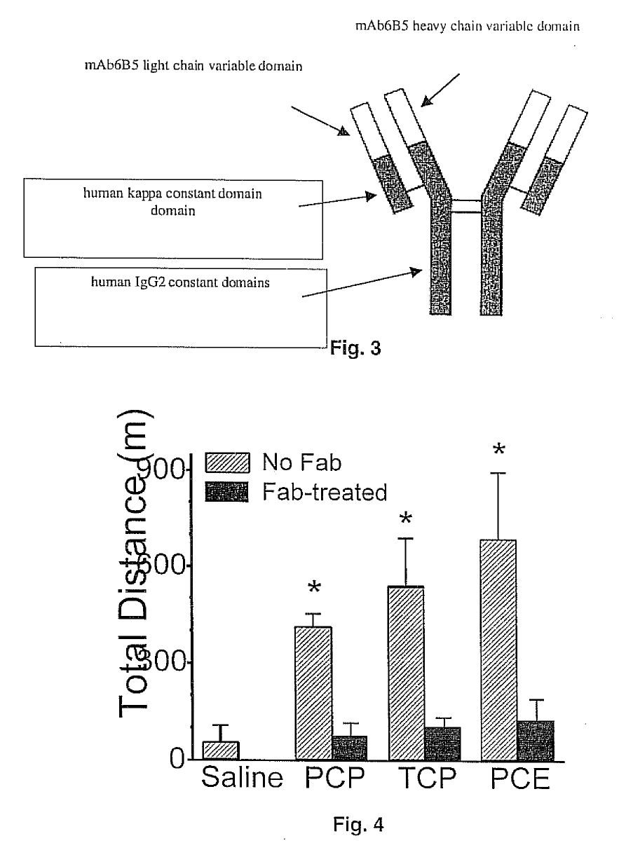

The present invention is directed to the use of monoclonal antibodies (mAb) to treatment drug abuse. Since mAb6B5 (of fully mouse origin) was not appropriate for human use, the present invention made mAb6B5 safe for human therapy. In order to accomplish this, the genes of mAb6B5 were engineered into a chimeric mouse/human antibody (ch-mAb6B5). This chimeric mouse/human antibody had the variable domains of mAb6B5 attached to the constant domains of human IgG2 heavy chain and kappa light chain (FIG. 3). For mAb6B5 to be a successful therapeutic, its antigen-binding site must remain intact since its effectiveness in reversing the effects of PCP is dependent on high PCP binding affinity (K_{D}=1.3 nM) and specificity. In addition, mAb6B5 has a unique tryptophan structure at its antigen-binding site that will help reduce its immunogenicity. X-ray crystallography of mAb6B5 Fab demonstrates that there is a tryptophan flap at the antigen-binding site that closes over the PCP molecule when it is in the antigen-binding site, much like a trash can lid. This unique structural feature is likely to prevent the formation of a PCP-like anti-idiotypic immune response.

Mouse/Human Chimeric mAb6b5

Of the three major strategies currently in use to produce therapeutic antibodies, only the chimeric approach is useful for pre-existing murine antibodies so that the antigen-binding site is not altered. For example, the procedure for producing a fully human antibody cannot be applied to a preexisting murine antibody. Although the humanization process uses protein sequences from an existing murine antibody, it has a major disadvantage in that it usually produces a loss of antigen-binding affinity and specificity. Only the chimeric mouse/human antibody retains the full native antigen-binding site, thus ensuring the antigen-binding affinity and specificity are not altered.

It can be argued that the humanized form is a better choice, since it is more human-like and will therefore be less immunogenic. Theoretically, this is true since a humanized antibody is approximately 95% human while a chimeric antibody is about 66% human (Clark, 2000). However, although studies have shown that both strategies decrease immune responses to the constant regions, neither strategies prevents the anti-idiotypic or anti-allotypic immune responses following repeated use. Both chimeric and humanized antibodies may provoke an anti-idiotypic response in approximately the same number of patients—12% (Kuus-Reichel et al., 1994). The FDA has indicated that chimeric, humanized and fully human monoclonal antibodies all have very low side effects due to immunogenicity.

To produce chimeric mouse/human mAb6B5, the mAb6B5 variable regions of the heavy and light chain were directly attached to the constant domains of the human IgG2 heavy chain and the kappa light chain, respectively. The IgG2 isotype was chosen. Of the five major isotypes of human antibodies, IgG is the preferred class to use for therapeutics because they are very stable, easily purified, have long-term stability during storage, and they have a long biological life in humans (˜21 days) in vivo. Within the human IgG class, there are four subclasses (IgG1-IgG4) that vary in their ability to trigger IgG effector functions, such as activating complement and binding Fc receptors. The subclasses IgG2 and IgG4, which are less efficient in activating effector functions, are less immunogenic. Furthermore, IgG2 has only one known allotype (sites of subtle genetic differences within an IgG subclass) making it less likely to produce an anti-allotypic immune response when administered to humans.

For the light chain human constant region, a kappa light chain was chosen. In humans and mice, there are only two isotypes for the light chain, kappa and lambda with kappa being the most prominent light chain in naturally occurring human antibodies (66%) and murine antibodies (95%). In studies to determine if different light chains affect the function of an antibody, no differences were shown between kappa and lambda light chains. Thus, this new chimeric form had the advantages of retaining the high affinity and specificity of native mAb6B5 PCP binding sites and yet being minimally immunogenic in humans because the constant regions are human.

Large Scale Production of Chimeric mAb6B5

The use of antibodies as therapeutic agents requires large-scale production, which has led to an increased demand for recombinant cell lines in which foreign genes can be transferred for expression of large quantities of protein. The cells most commonly used for this purpose are mammalian, since they will produce completely assembled and fully functional antibodies. A commonly used mammalian cell line for this purpose is Chinese hamster ovary cells (CHO). CHO cells are particularly suitable for the induction of gene amplification mechanisms, which increase protein production. The CHO cell gene amplification expression system was established by Alt et al. (1978) and was first used in the production of recombinant antibodies in 1991.

The scientific principle underlying the amplification system is described as follows. The genes for the light and heavy chains are transfected into dhfr-negative CHO cells in plasmids bearing a selectable marker gene, such as the neomycin resistance gene G418. A vector bearing the gene for dhfr is also co-transfected. Growing cells in G418 selects for cells transfected with the antibody genes. These cells are then cultured with increasing concentrations of methotrexate (MTX) to induce the gene amplification mechanism. MTX, a folic acid analog, binds and inhibits dhfr stoichiometrically, forcing cells to undergo genomic rearrangements and subsequent gene amplification of dhfr for survival. When the dhfr genes are amplified, neighboring genes (the co-transfected heavy and light chains) are often co-amplified. This method has been highly successful in producing cells lines that stably produce high levels of antibody (Page and Sydenham 1991; Peakman et al., 1994; Kunert et al., 2000).

The genes for the chimeric mAb6B5 antibody can also be inserted into plant cells for large scale production. Representative examples of plant systems that can be used are corn, tobacco or rice. However, other plant systems could be used.

The present invention was to convert murine anti-PCP antibody mAb6B5 into a form that can be used in humans as a medication for drug abuse. In order to be effective as a therapeutic agent, it was necessary for this new chimeric form to retain the same affinity and specificity of native murine mAb6B5 and yet be minimally, or non-immunogenic in humans.

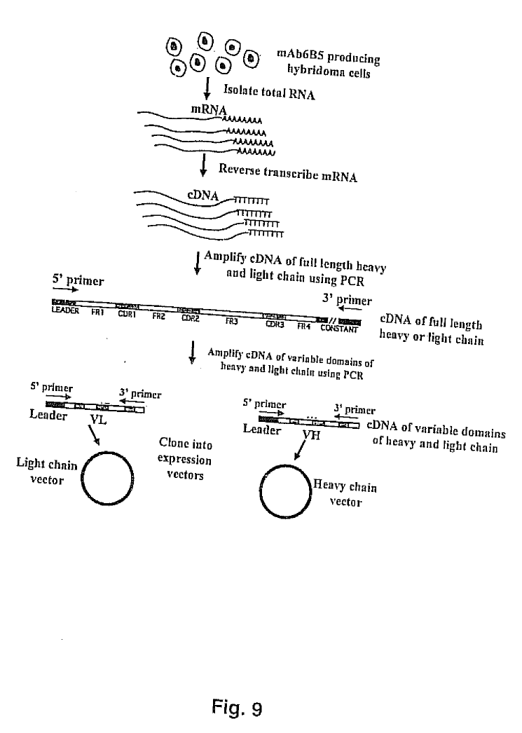

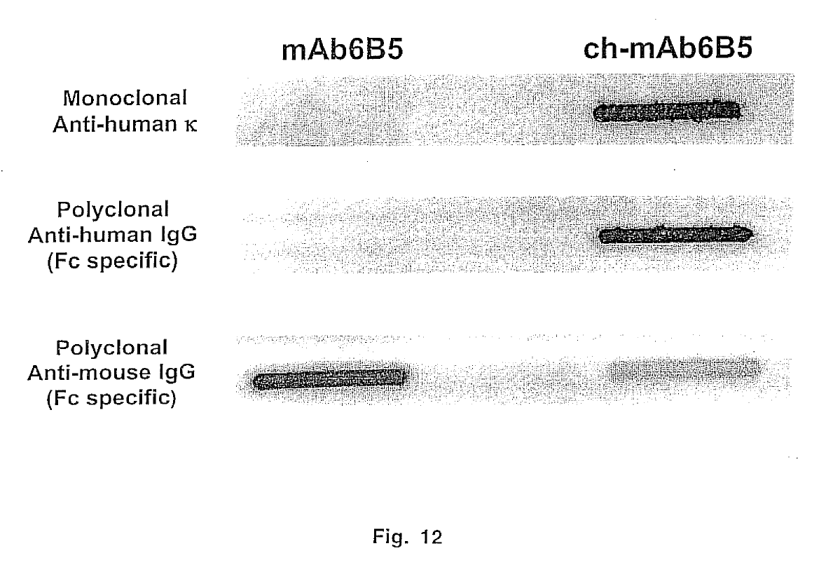

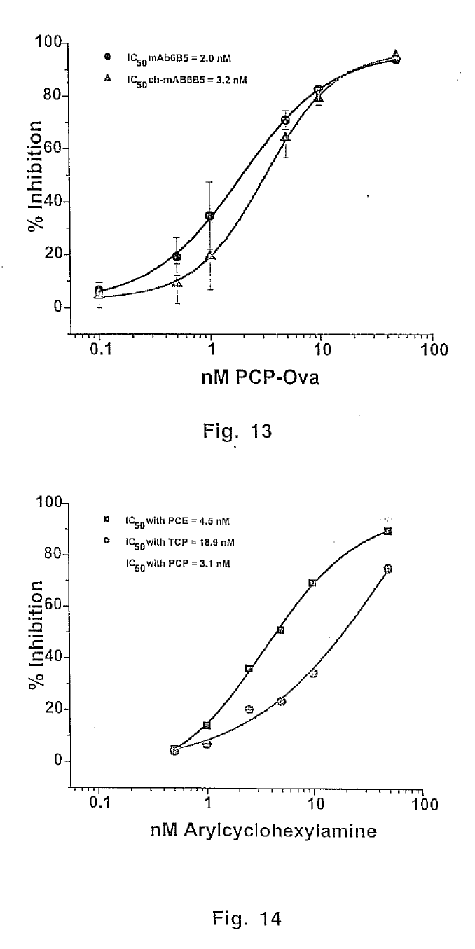

Hence, to accomplish this goal, mAb6B5 was genetically engineered into a chimeric (murine/human) antibody containing the PCP-binding site of mAb6B5 and the constant regions of human IgG₂ heavy chain and kappa light chain. The cDNA encoding V_{L} and V_{H} of mAb6B5 (PCP binding site) were ligated into light chain and heavy chain expression vectors containing genes for huCₖ or huC_{G2}, respectively (FIG. 9). These vectors were co-transfected into a non-producing murine myeloma cell line to produce a stable cell line expressing ch-mAb6B5. Sandwich ELISA testing and immunoslot blot demonstrated that the ch-mAb6B5 had human IgG heavy chain and kappa light chain (FIG. 12). Inhibition ELISA and radioimmunoassay (RIA) showed that ch-mAb6B5 not only bound to PCP and other arycyclohexylamaines, but it did so with the same affinity and specificity as native mAb6B5 (FIGS. 13 and 14). Taken together, these data indicated that the anti-PCP ch-mAb6B5 had functional characteristics of native mAb6B5, with the less immunogenic human constant region.

Further, since use of antibody as a therapeutic agent requires large-scale production, a widely used approach for large scale production is contemplated where the engineered genes for chimeric mAb6B5 will be expressed in dihydrofolate reductase (dhfr) negative Chinese hamster ovary (CHO) cells using the dhfr amplification system.

Thus, the present invention is directed to a chimeric mouse/human monoclonal antibody, which comprises human immunoglobulin constant domains and immunoglobulin variable domains of murine antibody. The human immunoglobulin constant domains are constant domains of human IgG heavy chain and human kappa light chain. Generally, the constant domain of human IgG heavy chain is human IgG2 heavy chain constant domain or IgG4 heavy chain constant domain. The chimeric light chain of the antibody, comprising the human immunoglobulin constant domain and the variable domain of murine antibody has the amino acid sequence of SEQ ID No. 16. Further, the chimeric light chain comprises DNA of SEQ ID No. 15. The chimeric heavy chain, which comprises of the human immunoglobulin constant domain and the variable domain of murine antibody has the amino acid sequence of SEQ ID No. 18. Further, the chimeric heavy chain comprises DNA of SEQ ID No. 17. Still further, the chimeric mouse/human monoclonal antibody is chimeric mAb6B5 antibody, which has the same chimeric light and heavy chains as described earlier.

Additionally, the mouse/human monoclonal antibody could also be used to treat arylcyclohexylamines drug abuse. Such a method comprises administering a pharmaceutically effective amount of the antibody to an individual, where the administration of the antibody reverses and/or reduces the adverse effects of arylcyclohexylamines drug abuse. The arylcyclohexylamines drugs include phencyclidine (PCP), 1-[1-(2-thienyl) cyclohexyl] piperidine (TCP) and N-ethyl-1-phenylcyclohexylamine (PCE). In one aspect, the monoclonal antibody that could be used to treat the drug abuse is chimeric mAb6B5 antibody.

Further, it is contemplated that the chimeric mouse/human monoclonal antibody could be used in a pharmaceutical composition. In such a case, the pharmaceutical composition comprises the novel chimeric antibody of the present invention and a pharmaceutically acceptable carrier generally known in the art. A person having ordinary skill in this art would readily be able to determine, without undue experimentation, the appropriate dosages and routes of administration for the chimeric antibody of the present invention. When used in vivo for therapy, the chimeric antibody of the present invention is administered to the patient or an animal in therapeutically effective amounts, i.e., amounts that decrease or reverse the adverse effects of arylcyclohexylamines drug abuse. It will normally be administered parenterally, preferably intravenously, but other routes of administration will be used as appropriate (e.g. intramuscular). The amount of chimeric mAb6B5 antibody administered will typically be in the range of about 0.01 mg/kg to about 100 mg/kg of patient weight. The schedule will be continued to optimize effectiveness while balanced against negative effects of treatment. See Remington's Pharmaceutical Science, 17th Ed. (1990) Mark Publishing Co., Easton Pa.; and Goodman and Gilman's: The Pharmacological Basis of Therapeutics 8th Ed (1990) Pergamon Press; which are incorporated herein by reference.

For parenteral administration, the chimeric antibody will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. The vehicle may contain minor amounts of additives such as substances that enhance isotonicity and chemical stability, e.g., buffers and preservatives.

The present invention is still further directed to an expression vector, which comprises DNA encoding human immunoglobulin light chain constant domain and immunoglobulin variable domain of murine antibody. All other aspects regarding the type of human immunoglobulin light chain constant domain, the DNA sequence as well as the amino acid sequence of the chimeric light chain expressed by the vector are as described earlier.

The present invention is also directed to an expression vector, which comprises DNA encoding human immunoglobulin heavy chain constant domain and immunoglobulin variable domain of murine antibody. All other aspects regarding the type of human immunoglobulin heavy chain constant domain, the DNA sequence as well as the amino acid sequence of the chimeric heavy chain expressed by the vector is as described earlier.

Additionally, the present invention is also directed to a host cell line comprising: a chimeric light chain expression vector and a chimeric heavy chain expression vector. All other aspects regarding the type of composition of the vectors and DNA sequences of the expression vectors are as described earlier. This host cell line could be a mammalian cell line or a plant cell line. Further, this host cell line also produces recombinant chimeric mouse/human monoclonal antibody which has the same amino acid sequence as described earlier. The recombinant antibody produced by the cell line is chimeric mAb6B5 antibody.

The present invention is directed to a method of producing recombinant chimeric monoclonal antibody. The method steps include amplifying the cDNAs of variable domains of murine monoclonal antibody, contructing chimeric light and heavy chain expression vectors comprising the amplified cDNAs and DNA encoding human immunoglobulin constant domain, co-transfecting the cell with the vectors, culturing the cell under conditions effective for expression of the recombinant antibody. The vectors can be co-transfected into a mammalian cell line or a plant cell line. The human immunoglobulin constant domains are as described earlier. The recombinant mouse/human monoclonal antibody produced by the method is chimeric mAb6B5 antibody.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

As used herein, the term “cDNA” shall refer to the DNA copy of the mRNA transcript of a gene.

The amino acids described herein are preferred to be in the “L” isomeric form. However, the residues in the “D” isomeric form can be substituted for any L-amino acid residue, as long as the desired functional property of immunoglobulin binding is retained by the polypeptide. NH₂ refers to the free amino group present at the amino terminus of a polypeptide. COOH refers to the free carboxy group present at the carboxy terminus of a polypeptide. In keeping with the standard nomenclature, J. Biol. Chem., 243:3552-59 (1969), abbreviations for amino acid residues may be used.

It should be noted that all amino acid residue sequences are represented herein by formulae whose left and right orientation is in the conventional direction of amino terminus to carboxy terminus. Furthermore, a dash at the beginning or end of an amino acid residue sequence indicates a peptide bond to a further sequence of one or more amino-acid residues.

As used herein, the term “PCR” refers to the polymerase chain reaction that is subject of U.S. Pat. Nos. 4,683,195 and 4,683,202 to Mullis, as well as improvements known in the art.

As used herein, “restriction endonucleases” and “restriction enzymes” refer to enzymes, each of which cut double-stranded DNA at or near a specific nucleotide.

The term “oligonucleotide”, as used herein, is defined as a molecule comprised of two or more ribonucleotides, preferably more than three. Its exact size will depend on many factors, which, in turn, depend upon the ultimate function and use of the oligonucleotide. The term “primer” used herein, refers to an oligonucleotide, whether occurring naturally (as in a purified restriction digest) or produced synthetically, and which is capable of initiating synthesis of a strand complementary to a nucleic acid when placed under appropriate conditions, i.e., in the presence of nucleotides and an inducing agent, such as a DNA polymerase, and at a suitable temperature and pH. The primer may either be single-stranded or double-stranded and must be sufficiently long to prime the synthesis of the desired extension product in the presence of inducing agent. The exact length of the primer will depend upon many factors, including temperature, sequence and/or homology of primer and the method used. For example, in diagnostic applications, the oligonucleotide primer typically contains 15-25 or more nucleotides, depending upon the complexity of the target sequence, although it may contain fewer nucleotides.

The primers used herein are selected to be “substantially” complementary to particular target DNA sequences. This means that the primers must be sufficiently complementary to hybridize with their respective strands. Therefore, the primer sequence need not reflect the exact sequence of the template. For example, a non-complementary nucleotide fragment (i.e., containing a restriction site) may be attached to the 5′ end of the primer, with the remainder of the primer sequence being complementary to the strand. Alternatively, non-complementary bases or longer sequences can be interspersed into the primer, provided that the primer sequence has sufficient complementary bases with the sequence to hybridize therewith and form the template for synthesis of the extension product.

As used herein “vector” may be defined as a replicable nucleic acid construct, e.g., a plasmid or viral nucleic acid. Vectors may be used to amplify and/or express nucleic acid encoding the chimeric light and heavy chain of mouse/human monoclonal antibody. An expression vector is a replicable construct in which a nucleic acid sequence encoding a polypeptide is operably linked to suitable control sequences capable of effecting expression of the polypeptide in a cell. The need for such control sequences will vary depending upon the cell selected and the transformation method chosen. Generally, control sequences include a transcriptional promoter and/or enhancer, suitable mRNA ribosomal binding sites, and sequences, which control the termination of transcription and translation. Methods, which are well known to those skilled in the art, can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control the transcription of the gene.

In general, expression vectors containing promoter sequences, which facilitate the efficient transcription of the inserted DNA fragment, are used in connection with the host.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

The Antigen-Binding Fragment (Fab) of mAb6b5 Reverses the Locomotor Effects Induced by an Overdose of PCP and Other Arylcyclohexylamines

These experiments were designed to test the effectiveness of mAb6B5 as a pharmacokinetic antagonist in animal models of human overdose of PCP and PCP-like drugs (i.e. TCP and PCE) (Hardin et al., 1998). If a single dose of Fab reverses the toxic effects of multiple members of the arylcyclohexylamine drug class, then mAb6B5 could be used as an immunotherapeutic agent for the treatment of most of the members of this dangerous class of drugs.

To test mAb6B5 Fab, male Sprague-Dawley rats were administered intravenously at 3 mg/kg of PCP, TCP or PCE. Thirty minutes after drug administration, mAb6B5 Fab or saline was administered i.v. (FIG. 4). Fab was used instead of the intact IgG molecules because it was hypothesized that Fab would be the best drug overdose treatment since Fab is rapidly cleared. The mAb6B5 Fab has essentially the same K_{D} value to the intact IgG (1.8 nM vs. 1.3 nM, respectively). The Fab dose was calculated on the basis of stoichiometric mole-equivalents (mol-eq) of binding sites to the mole dose of the drug. For example, if a 300 g rat received a 4 mg/kg dose of PCP (MW=243), a 1.0 mol-eq dose of mAb6B5 Fab (50 kD) would be 185 mg.

To assess pharmacological changes, video tracking and digitized motion analysis were used to evaluate the behavioral parameters “distance traveled” and “total movement” over a 2.5 hr period. These studies demonstrate that mAb6B5 is highly effective in reversing the locomotor effects of PCP and other arylcyclohexylamines (FIG. 4). To put these changes in perspective, the average distance traveled by a rat given saline control during the 2.5 hr test period was about the distance of a football field (one seventeenth of a mile). When rats were given PCR, they traveled almost one-half mile on the average and one animal traveled 0.9 miles. After Fab treatment this distance was reduced to about two football fields. This distance was not significantly different from the saline control value.

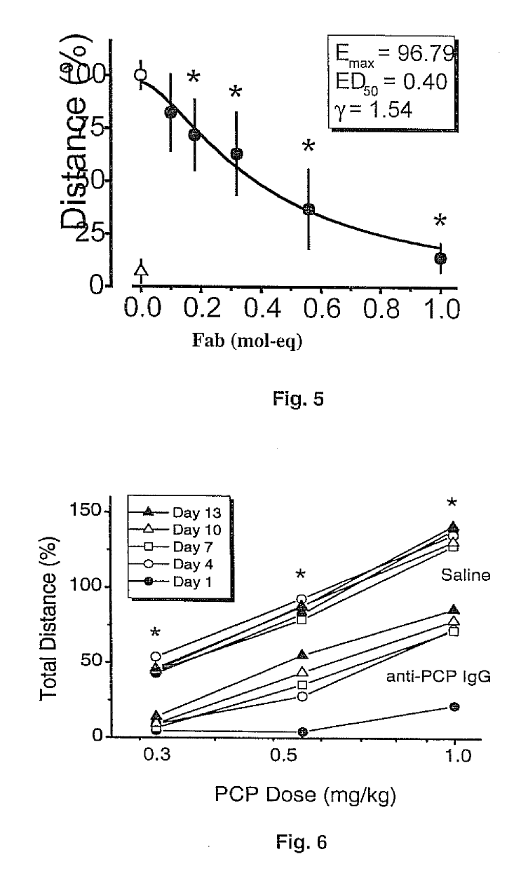

To determine the amount of mAb6B5 Fab needed for optimal treatment of overdose, a Fab dose-response curve was determined for its effects on a dose of 3 mg/kg PCP (FIG. 5). Five doses of Fab ranging from 0-1.0 mol-eq were administered to rats thirty minutes after the PCP dose (closed circles). Control groups received saline-saline (open triangle) or saline after PCP (open circle). Total distance traveled was measured and expressed as a percentage of the response to PCP without Fab treatment (i.e., 100% response). These data indicate that mAb6B5 Fab reverses the effect of PCP in a dose-dependent manner. A complete inhibition of the behavioral effects of PCP is achieved at an equimolar amount of Fab, while a 50% reduction in maximal response is achieved with a dose of Fab that is 40% equimolar to the dose of PCP. These data demonstrate that mAb6B5 effectively treats the abuse of a class of drugs, the arycyclohexylamines, as well as a single drug, PCP.

### EXAMPLE 2

A Single Dose of mAb6b5 IgG Provides Long-Term Reductions in PCP-Induced Locomotor Effects

These studies tested the hypothesis that a single dose of intact mAb6B5 IgG can provide long-term protection against the effects of repeated PCP administration in rats (Hardin et al., 2002). Male Sprague-Dawley rats received i.v. treatments of saline, non-specific bovine IgG (1.0 mg/kg) or mAb6B5 IgG (1.0 mg/kg) on day 1. The rats were then challenged with escalating doses of PCP (0.32, 0.56, and 1.0 mg/kg) spaced 90 minutes apart. This dosing regimen was repeated on days 4, 7, 10 and 13 (totaling 15 PCP doses) (FIG. 6). The experiments were terminated after two weeks because of cannulae failure, which occurred at periods longer than two weeks dosing. In terms of human PCP use, this regimen would equate to about 45 recreational doses (at 5 mg/dose) over a long period of time (at least 2-3 months).

Locomotor activity (the total distance traveled) for each rat was measured using the Noldus EthoVision behavior imaging system. In both the saline and non-specific IgG control groups, escalating doses of PCP produced a linear and reproducible dose-dependent locomotor response. There were no differences in the locomotor responses between the saline and non-specific IgG control groups so only the saline control group is shown in FIG. 6. In contrast to the control group, on the first day of PCP challenge, the mAb6B5 IgG treatment completely blocked PCP effects. Over the next four sessions of PCP challenges (days 4, 7, 10 and 13), the protective effects of mAb6B5 equilibrated to a constant 50% reduction in effects. These results show that a single dose of mAb6B5 IgG protects against repeated PCP challenges even after 2 weeks, and even when the antibody binding capacity should have been “saturated” on the second day of dosing.

### EXAMPLE 3

mAb6B5 IgG Provides Long-Term Neuroprotection

These studies demonstrated that a single large dose of mAb6B5 IgG provides long-term reductions in brain PCP concentrations, despite continuous PCP administration (Proksch et al., 2000). Rats were implanted with s. c. osmotic minipumps filled to deliver PCP at a rate of 18 mg/kg/day. Steady-state PCP concentrations were achieved at less than 24 hr since the PCP half-life (t_{1/2}) is 4 hr. At 24 hr after implantation of the pumps, a mol-eq dose of a mAb6B5 IgG binding sites was administered intravenously. The PCP infusion continued for up to 27 days (approximately one month). At selected time points after administration of the antibody, brain, serum and testis PCP concentrations were measured in groups of animals.

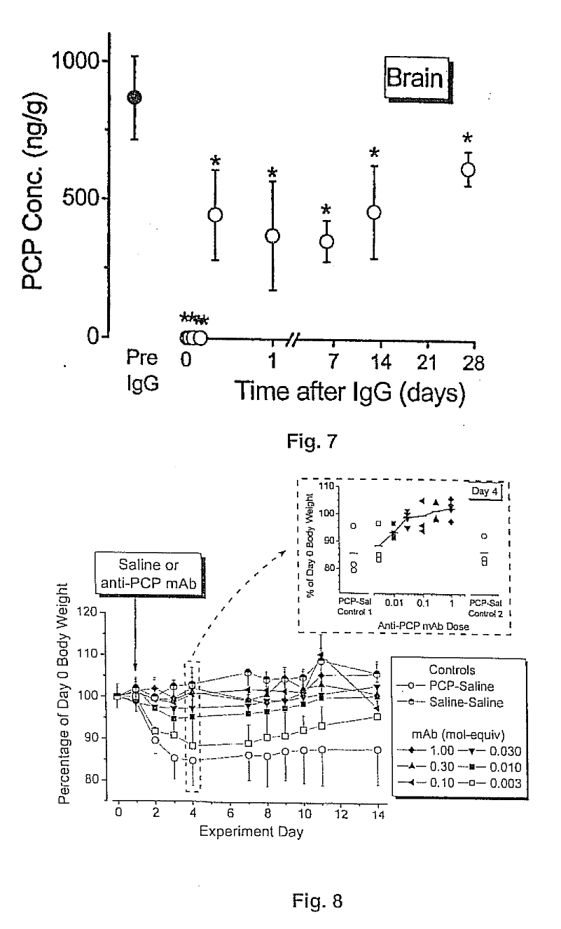

After mAb6B5 administration, serum PCP concentrations rapidly increased approximately 300-fold, while there was a complete removal of PCP from the brain within 15 min, which persisted for the first 4 hr (FIG. 7). In addition, the antibody consistently decreased brain PCP concentrations by an average of 53% from 8 hr to 14 days. Even after 27 days of constant PCP infusion, the PCP concentration in the brain was still decreased by 28% (*P<0.5, for all values from 8 hours to 27 days) compared to the steady state pre-mAb6B5 control values. These results indicate that mAb6B5 IgG can protect the brain for at least four weeks after one dose, even when the antibody binding capacity should have been saturated within the first few hours of continuously administered PCP, and the drug was being replaced at a rate of 15% of the body burden per hour.

### EXAMPLE 4

A Single Low Dose of mAb6B5 IgG Provides Long-Term Protection Against PCP's Adverse Health Effects

This series of experiments assessed the dose-dependent effects mAb6B5 IgG on measures of health and behavior (Laurenzana et al., 2003). Rats were implanted with s.c. osmotic minipumps filled to deliver PCP at a rate of 18 mg/kg/day. Baseline PCP-induced locomotor activity was assessed (24 hr after start of PCP infusion), and then saline or various doses of mAb6B5 IgG were administered i.v. The doses of mAb6B5 ranged from 1 mol-eq of serum PCP concentration at steady state (1.53 g/kg of mAb6B5 IgG), down to 0.003 mol-eq (0.005 g/kg).

On a daily basis, rats were assessed on the amount of food eaten, body weight, general health, and evidence of chromodacryorrhea (“bloody tears” or red lacrimal secretions), an indicator of PCP-induced stress. Assessment of locomotor activity on day 1 after mAb6B5 IgG administration showed that mAb6B5 IgG treatment immediately reduced locomotor activity in all mAb6B5 IgG treatment groups, except for the lowest dose, 0.003 mol-eq. The dose-dependent effects of mAb6B5 IgG on body weight produced some of the most interesting and profound effects (FIG. 8). Rats receiving 0.01 to 1.0 mol-eq of antibody did not have a significant reduction in body weight, while the PCP-saline group and the group receiving the lowest antibody dose (0.003 mol-eq) showed a significant decrease in body weight compared with the other treatment group (p<0.05). PCP-induced reductions in feeding and drinking behavior obviously contributed to the weight loss in these groups. But their eating behavior normalized by day 4, probably secondary to the development of tolerance to the drug. This allowed these rats to better maintain their health and well-being. However, they did not regain all of the weight they lost during days 2 and 3 of the study. The body weights of PCP-treated rats that received anti-PCP mAb at doses ranging from 0.01 to 1 mol Eq were not significantly different from control rats that received saline infusion (without PCP) and a saline treatment on day 1 (saline-saline group, FIG. 8). The PCP-induced weight loss achieved maximum on about day 4 of the experiment. On this day, the mAb showed a dose response for protection against weight loss (FIG. 8, inset). On experiment days 4 through 7, the rats in the PCP-saline and PCP-0.003 mol Eq groups showed the most profound PCP-induced adverse effects. 25% of the rats died or had to be sacrificed for humane reasons. This was observed again when the PCP-saline control experiment was repeated in a separate group of rats (1 of 4 of the animals died as a result of PCP administration).

These results are profoundly important because they show that extremely low doses of mAb6B5 IgG, which is only 1:100 mol-eq of the body burden of PCP on day 1, can offer long-term protection against adverse health effects of PCP. This is also a reasonable model of the adverse health effects humans experience during their binge usage of stimulants like PCP. Indeed, humans often repeatedly self-administer stimulants at great cost to their health status. This is a point that is often ignored in the development of medications for treating drug abuse. These studies show that in rats, doses of mAB6B5 as low as 15 mg/kg (equivalent to about a 1 gm total dose in a 70 kg human) are effective at improving and stabilizing the health of the animals (i.e., reduced behavioral effects, no weight loss and reduced levels of animal stress), even when they continue to use life-threatening doses of PCP. Additionally, the dose of PCP that was administered to the rats has a human equivalent of 1.26 grams of PCP per day for 2 weeks (18 mg/kg.times.79 kg human=1.26 g/day). These findings have implications for the usefulness of this antibody as a medication for the people abusing PCP and other arylcyclohexylamines.

The fact that mAb6B5 IgG, at very low doses, can provide long-term protection against the adverse effects of a PCP makes this antibody a viable and economically feasible immunotherapy for the treatment of PCP and PCP-like drug abuse. The present invention further engineers mAb6B5 into a form that can be used safely in humans while retaining its impressive characteristics as an immunotherapeutic agent.

### EXAMPLE 5

Cell Lines Used

The anti-PCP mAb6B5 hybridoma cell line was produced as described in Valentine et. al., 1994. Briefly, BIO.H-2^{a}H—Y^{b} mice were immunized with the PCP metabolite PCHAP covalently bound to bovine serum albumin. After fusion of spleen cells from the mice with a myeloma cell line, hybridomas secreting anti-PCP antibodies were identified by using an enzyme-linked immunosorbant assay with PCHAP coupled to ovalbumin. Wells with a positive reaction to PCP were subcloned to monoclonality. The murine non-producing myeloma cell lines P3x63-Ag8.653 (P3X) (CRL 1580) and Sp2/0 (CRL 1581) were obtained from the American Type Culture Collection (Rockville, Md.). All cell lines were maintained at 37° C. under 10% CO₂ in DMEM (Gibco, Carlsbad, Calif.) supplemented with 10% FCS (HyClone, Logan, Utah).

Engineering and Expression of Chimeric Mouse/Human mAb6B5

The mAb6B5 was engineered into a mouse/human chimeric antibody that consisted of the variable domains of the mAb6B5 heavy chain (V_{H}) and light chain (V_{L}) (˜12.5 kD each) attached to the constant domains of human IgG2 (˜37.5 kD) and kappa (˜12.5 kD), respectively. Briefly, the strategy to construct the genes for chimeric mAb6b5 light chain and heavy chain involves: (1). Cloning the cDNA of mAb6B5 V_{L} and V_{H}, including their respective leader sequences by RT-PCR. The N-terminal leader sequences were necessary to insure proper assembly and secretion of chimeric mAb6B5. (2). The V_{L} and V_{H} cDNA were ligated into expression vectors-one for light chain (LC), containing the sequences for huCₖ and one for the heavy chain (HC), containing the sequences for huC_{G2}. (3). The LC and HC expression vectors were co-transfected into a non-producing murine myeloma cell line to express chimeric mAb6B5. With this structure, the chimeric mAb6B5 retains the unique antigen-binding properties of native mAb6B5 and becomes significantly less immunogenic to humans. The genes of the chimeric mAb6B5 were engineered into expression vectors and expressed in a mammalian expression system designed to produce large amounts of the antibody. The effectiveness of native mAb6B5 in reversing adverse effects of PCP and other arylcyclohexylamines, and 2) the pharmacokinetic properties of the chimeric mAb6B5 compared to native mAb6B5 can be shown.

The cDNA for mAb6B5 V_{L} and V_{H} had been cloned and sequenced without the leader (L) sequences or constant regions (Lim, 1998). Since most of the sequence that was required to amplify the appropriate sequence of each chain (the leader sequence through the J-C junction) was unknown, the full-length LC and HC cDNA from the leader sequence (LS) to the C-terminus were cloned. A polymerase chain reaction (PCR)-based cloning strategy was used to produced the chimeric mouse/human antibodies (Coloma et al., 1992; Morrison, 1994).

In this method, the cDNA for the V_{H} and V_{L} of an antibody are cloned from the antibody producing hybridoma cells using reverse-transcription and two rounds of PCR (FIG. 9). The challenge of PCR-based cloning of monoclonal antibodies is that their cDNA sequence is not known, and PCR requires the knowledge of the DNA sequence in order to produce the PCR primers. Designing primers for the C-terminal end of both heavy and light chains is relatively straight forward, because the C-termini consist of the “constant” regions. However, designing primers for the N-termini (the variable domains) is more difficult. This strategy takes advantage of a sequence of DNA that is located 5′ of the variable domain on both the heavy and light chains. This sequence encodes for a leader or signal peptide that is at the N-terminus of newly translated light and heavy chains (FIG. 9). The leader peptide directs the heavy or light chains into the endoplasmic reticulum in preparation for secretion from the cell. Upon insertion into the ER, the leader peptide is removed from the polypeptide. The leader sequences of murine IgG1 and kappa chains do not vary much.

Based on the conservancy of the leader sequences, sets of degenerate 5′ primers were developed that prime through the leader sequence to amplify the murine IgG1 heavy chains and kappa light chains (Tables 1 and 2, SEQ ID Nos. 1-8). This approach of priming through the leader sequence has an advantage in that the leader sequence is removed from the mature antibody molecule, therefore variations in sequence introduced through priming with degenerate primers will not affect antibody affinity. Once the full-length cDNA of the heavy and light chains are PCR-amplified and cloned, they are sequenced. From the sequences primers can be designed to amplify only the V_{H} and V_{L} regions. After PCR amplification of the V_{H} and V_{L}, the cDNA can be cloned and inserted into expression vectors.

TABLE 1
5′ primers used for amplification of leader region
of murine IgG1

| | Primer Sequence |
|---|---|
| PCR7 Primer | (SEQ ID NO) |
| MHALT1.RV | 5′-GGGGATATCCACCATGGrATGsAGCTGkGTmATsC |
| | TCTT-3′ |
| | (SEQ ID NO. 1) |
| MHALT2.RV | 5′-GGGGATATCCACCATGrACTTCGGGyTGAGCTkGG |
| | TTTT-3′ |
| | (SEQ ID NO. 2) |
| MHALT3.RV | 5′-GGGGATATCCACCATGGCTGTCTTGGGGCTGCTCT |
| | TCT-3′ |
| | (SEQ ID NO. 3) |

TABLE 2
5′ primers used for amplification of leader region
of murine kappa chain

| | Primer Sequence |
|---|---|
| PCR Primer | (SEQ ID NO) |
| MLALT1.RV | 5′GGGGATATCCACCATGGAGACAGACACACTCCTGCT |
| | AT-3′ |
| | (SEQ ID NO. 4) |
| MLALT2.RV | 5′-GGGGATATCCACCATGGATTTTCAGGTGCAGATTT |
| | TCAG-3′ |
| | (SEQ ID NO. 5) |
| MLALT3.RV | 5′-GGGGATATCCACCATGrAGTCACAkACyCAGGTCT |
| | TyrTA-3′ |
| | (SEQ ID NO. 6) |
| MLALT4.RV | 5′-GGGGATATCCACCATGAGGkCCCCwGCTCAGyTyC |
| | TkGGr-3′ |
| | (SEQ ID NO. 7) |
| MLALT5.RV | 5′-GGGGATATCCACCATGAAGTTGCCTGTTAGGCTGT |
| | TG-3′ |
| | (SEQ ID NO. 8) |

¹EcoRV sites are underlined.
Ribosome binding sites are in bold.
Degeneracies are indicated by lower case letters.

Cloning of Full Length Heavy and Light Chain cDNA

Total RNA was isolated from mAb6B5 hybridoma cells with a RNeasy minikit (Qiagen, Valencia, Calif.) and used for first strand cDNA synthesis with M-MLV reverse transcriptase and an oligo (dT)₁₅ primer. The resulting cDNA was used in a first round PCR reaction to amplify full-length cDNA of mAb6B5 heavy and light chains.

The mAb6B5 HC and LC cDNA were amplified using sets of degenerate primers based on the conservancy of leader sequences of murine IgG1 and kappa chains (Coloma et al., 1992; Morrison, 1994). The 5′ primers used for this first round of PCR were as shown in Tables 1 and 2. For the first round of PCR, the 5′ primers consisted of a set of degenerate 5′ primers to prime through the L sequences. For the murine IgG 1 heavy chain there was a set of 3 primers (SEQ ID Nos. 1-3); for the murine kappa light chain there was a set of 5 primers (SEQ ID Nos. 4-8). The isotype of the mAb6B5 heavy and light chain had been previously tested and shown to be IgG1 with a kappa light chain. The 3′ primers for the heavy chain and the light chain were based on conserved sequences of the C-termini of murine IgG1 and kappa chains listed in the GenBank database of the National Center for Biotechnology Information (NCBI). All primers (both 5′ and 3′) were made by Integrated DNA technologies, Inc (Coralville, Iowa) and were designed with a specific restriction enzyme site at their 5′ end flanked by three additional bases (see Table 1 and 2). The three additional bases are added to protect the restriction site and facilitate enzyme digestion. The restriction sites are chosen and added to allow the insertion of the amplified cDNA into expression vectors in a specific orientation. Pfu polymerase (Stratagene, La Jolla, Calif.) was used in all PCR reactions, because it has proofreading capability and therefore very high fidelity.

The PCR amplifications were carried out in a GeneAmp PCR system 9700 (Applied Biosystems, Foster City, Calif.) under the following conditions for the kappa light chain are as follows: 35 cycles of 94° C. for 1 min (denaturation), 57° C. for 1 min (annealing), 72° C. for 3 min (extension) followed by a final 10 min cycle of extension at 72° C. The conditions for amplification of the IgG1 heavy chain were similar except the annealing temperature was 62° C. Three separate heavy chain PCR reactions were conducted, each using one of the three degenerate 5′ primers (which are labeled MHALT1.RV, MHALT1.RV, MHALT2.RV) and the same 3′ primer. Ethidium bromide staining of the gel reveals a band of the appropriate size for a full length IgG1cDNA sequence [˜1500 base pairs (bp)] amplified with the MHALT1.RV primer. The other 5′ primers, (MHALT2.RV) and (MHALT3.RV), produced no bands.

The reaction conditions with the (MHALT1.RV) primer were then optimized with increased amounts of magnesium chloride. Amplification of the 1500 bp cDNA with 5′ primer MH1 was much more efficient with 2.5 mM MgCl₂. This band was excised from the gel, purified and frozen at −20° C. to use for cloning.

The PCR products for the light chain yielded a strong cDNA band of the appropriate size (˜700 bp) with 5′ primer MLALT5.RV. This band was also gel purified and frozen for use in cloning.

To determine if MHALT1.RV and MLALT5.RV cDNAs were the full-length cDNAs for mAb6B5 heavy and light chains, they were cloned into a vector and sequenced. For cloning, each cDNA was ligated into pcDNA3.1 (Invitrogen, Carlsbad, Calif.), an expression vector that may be used in the future to express native murine mAb6B5 in mammalian cells. Using the restriction sites encoded into the MHALT1.RV and MLALT5.RV cDNAs via the PCR primers, the MHALT1.RV and MLALT5.RV were inserted into the pcDNA3.1+separately. The ligation reactions were used to transform E. coli Dha competent cells (Invitrogen). Transformants (bacteria which incorporated the vector) were selected by plating the bacteria onto LB plates containing 100 μg/ml ampicillin. For each of the two constructs (MHALT1.RV and MLALT5.RV) five clones were selected and analyzed for the presence of the inserted cDNA by restriction enzyme digestion followed by agarose gel electrophoresis. All 5 clones of MHALT1.RV and MLALT5.RV were sequenced. Comparison of the consensus sequences of MHALT1.RV and MLALT5.RV to sequences in the NCBI GenBank database demonstrated that MHALT1.RV is a full-length cDNA for a murine IgG1 heavy chain. As expected, the sequence for the constant domains is a perfect match for other murine IgG1 heavy chains and the sequence for the variable domain, although conserved, varies from other IgG1 in the expected regions. Similarly, MLALT5.RV is a full-length cDNA for a murine kappa light chain. Like MHALT1.RV, the sequence for the constant domain is a perfect match for other kappa chains; the variable domain differs slightly in the appropriate areas. Thus, full-length cDNAs for the heavy and light chains of mAB6B5 were successfully cloned.

Construction of Chimeric mAb6B5 (ch-mAb6B5) LC Expression Vector (PLC-huCₖ)

Based on the sequence of the cloned mAb6B5 LC, the following primers were designed to amplify the V_{L} of mAb6B5: 5′ primer-5′CCCGCTAGCCACCATGAAGTTGCCTGTTAGGCTGTTG 3′ (SEQ ID No. 9, NheI site is underlined; the ribosome landing site is bold) and 3′primer-5′TATAGCGGCCGCAGTTTTTATTTCCAGCTTG3′(SEQ ID No. 10, NotI site is underlined). The PCR amplification conditions were as follows: 35 cycles at 94° C. for 1 min (denaturation), 62° C. for 1 min (annealing), 72° C. for 3 min (extension) followed by a final 10 min cycle of extension at 72° C. The amplified mAb6B5 V_{L} was cloned directly into expression vector pLC-huCₖ, which was provided by Dr. Gary McLean (University of British Columbia, Vancouver).

The pLC-huCₖ is a light chain expression vector containing the cDNA sequences of huC_{K} and the strong CMV promoter (McLean et al., 2000) Using the NheI and NotI sites, the amplified mAb6B5 was directionally ligated into pLC-huC_{K} immediately upstream of the huC_{K} genes (with no intervening sequence) and directly downstream of the CMV promoter. This configuration enables transcription of the open reading frame downstream of the CMV promoter to produce a cDNA of ch-mAb6B5 LC. The ligated products were used to transform DH5α competent cells (Invitrogen), and multiple clones were sequenced to confirm the sequence accuracy of ch-mAb6B5 LC.

Construction of ch-mAb6B5 HC Expression Vector (pHC-huC_{G2})

Based on the sequences of the cloned mAb6B5 heavy chain, the following primers were designed to amplify the V_{H} of mAb6B5: the 5′ primer 5′GGGGATATCCACCATGGAATGCAGCTGTGTAATGCTCTT3′, SEQ ID NO. 11, EcoRV site is underlined) was similar to SEQ ID No. 1, except r at position 18 was A, s at position 22 was C, k at position 28 was T, m at position 31 was A and s at position 34 was G. The 3′ primer was 5′GGGGCTAGCTGAGGAGACTGTGAGAGTGGT3′ (SEQ ID No. 12, the NheI site is underlined). The PCR amplification conditions were same as the amplification of V_{L} except that the annealing temperature was 59° C. The amplified mAb6B5 V_{H} was cloned into pPCR-Script Amp cloning vector (Stratagene, La Jolla, Calif.) for sequencing and then subcloned into mammalian expression vector pAH46 18 provided by Dr. Sherie Morrison (UCLA, Los Angeles).

Expression vector pAH4618 was designed as immunoglobulin heavy chain expression vector. It contains the murine heavy chain promoter, the genomic sequences of huC_{G2} and the hisD selectable marker (Coloma, 1992). Using the EcoRV and NheI sites, mAb6B5 V_{H} was ligated into the vector directly upstream of the huC_{G2} genomic sequences and directly downstream of the murine heavy chain promoter. The ligation products were used to transform HB101 competent cells (Invitrogen). Clones containing the ch-mAb6B5 HC construct were identified by restriction enzyme digestion and plasmid preps were performed to purify the ch-mAb6B5 HC expression construct.

The ch-mAb6B5 HC expression construct was linearized with Pvul and co-transfected into Sp2/0 murine myeloma cells described below along with a LC expression construct. Transfected cells were grown in the presence of 5 mM histidinol (Sigma, St. Louis, Mo.) and clones expressing fully assembled ch-mAb6B5 were screened using a sandwich ELISA as described later. No positive clones were found. Thus, to screen for the transcription of mRNA for the mAb6B5 LC or HC, total RNA was isolated from a pool of transfected cells and RT-PCR was performed to amplify the chimeric LC and HC. The chimeric HC but not the LC was amplified. The 5′ primer used to amplify the ch-mAb6B5 (5′ GGGGCTAGCCACCATGGAATGCAGCTGTGTAATGCTCTT3′, SEQ ID NO. 13) was the same 5′ primer used to amplify mAb6B5 V_{H}, described earlier, except a NheI site (underlined) replaced the EcoRV site. The 3′ primer—5′ GGGCTCGAGTCATTTACCCGGAGACAGGGAG 3′ (SEQ ID No. 14) was designed based on the conserved sequences at the C-terminus of huC_{G2} listed in the NCBI GenBank. It includes a XhoI restriction site (underlined). The HC expression vector used to produce chimeric mAb6B5 was constructed using the expression vector pHC-huC_{G2} provided by Dr. Gary McLean (McLean et al., 2000).

This vector contains the cDNA sequences of huC_{G2}, and the CMV promoter. The full-length cDNA of ch-mAb6B5 HC was cloned into pHC-huC_{G2} after removing the existing huC_{G2} by restriction digestions with NheI and XhoI. The mAb6B5 HC cDNA was directionally inserted into the vector immediately downstream of the CMV promoter. The ligation products were used to transform DHα competent cells (Invitrogen) and multiple clones of the ch-mAb6B5 heavy chain construct were sequenced to confirm that the open reading frame of ch-mAb6B5 HC was correct.

Thus, two sets of mammalian expression vectors specifically designed to express functional recombinant antibodies were used. The first set of expression vectors was constructed (Coloma, 1992) and provided by Dr. Sherie Morrison (UCLA, Los Angeles). The HC vector pAH4618 contained the genomic sequences for huC_{G2} and the LC vector pAG4622 contained the genomic sequences for huC_{K}. Both utilized the murine heavy chain promoter. These vectors were members of a family of expression vectors produced specifically to facilitate the cloning and expression of immunoglobulin variable regions cloned by PCR (Morrison, 1994; Coloma et al., 1992). The V_{L} and V_{H} were directionally inserted into the appropriate vectors upstream of the constant region sequences. Both LC and HC constructs were linearized and co-transfected into murine myeloma cell lines P3X and Sp2/0. Three transfections were performed to establish a stable cell line expressing assembled ch-mAb6B5. However, only the HC was expressed in Sp2/0 cells. The light chain was never expressed. Therefore, only the full-length heavy chain was cloned and sequenced using RT-PCR.

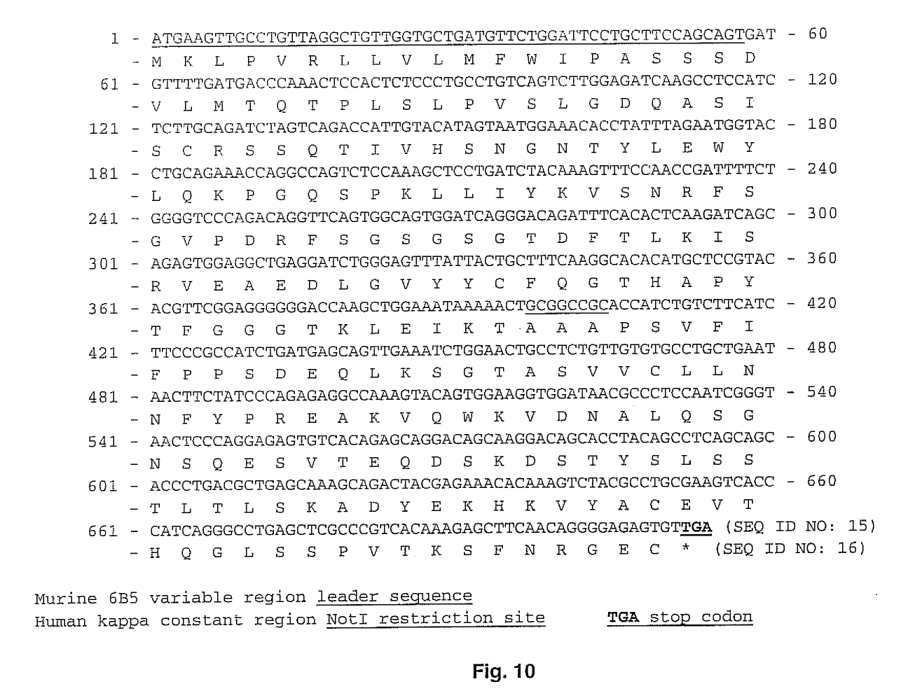

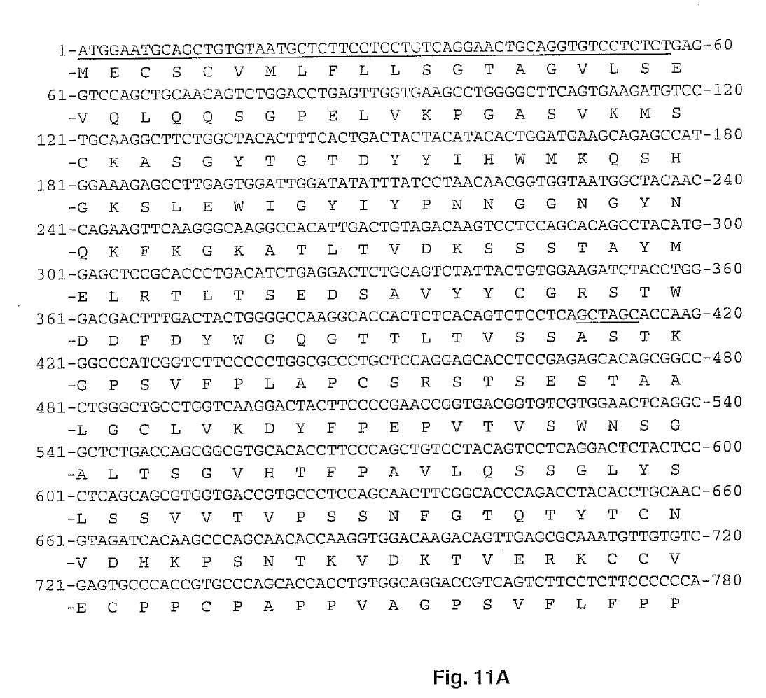

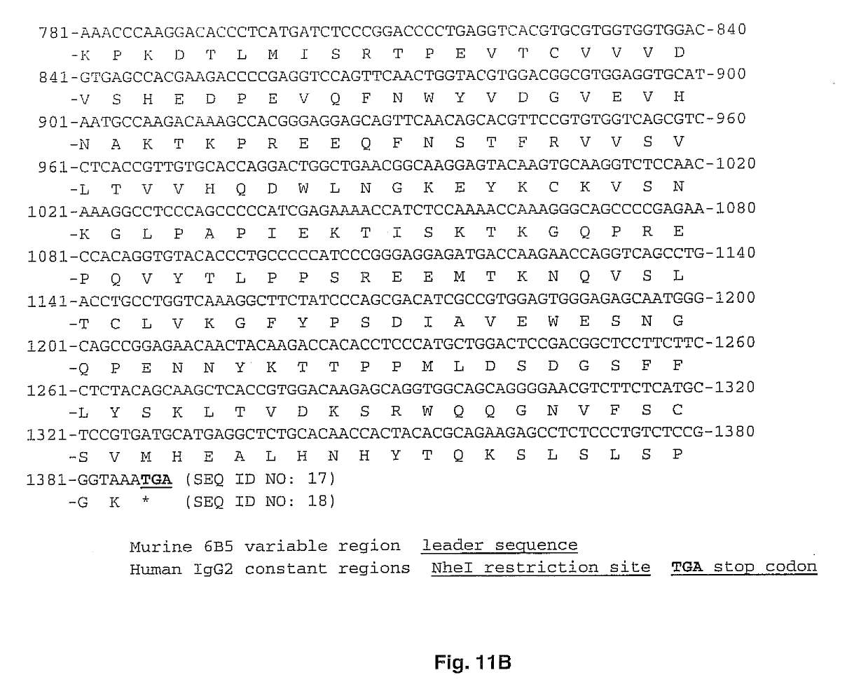

The ch-mAb6B5 antibody was successfully expressed using the second set of immunoglobulin expression vectors. Heavy chain vector pHC-huC_{G2} and light chain vector huC_{K} were provided by Dr. Gary McLean (University of British Columbia, Vancouver). As described by McLean et al, 2000, these vectors contain the strong viral CMV promoter and the cDNA of huC_{G2} and huC_{K}. The CMV promoter abrogates the requirement for intronic sequences and allows the use of cDNA instead of larger genomic sequences of human constant regions. These vectors have the advantages of being relatively small in size and a high copy number. The cDNA of mAb6B5 V_{L} was ligated into expression vector pLC-huCₖ immediately upstream of the cDNA of huCₖ and downstream of the CMV promoter. For the HC construct a slightly different strategy was used. The cDNA of full-length ch-mAb6B5 heavy chain (previously cloned from the transfections and expression with the Morrison HC vector, pAG4618) was ligated into vectorpHC-huC_{G2} downstream of the CMV promoter—after first removing the pre-existing huC_{G2} sequences from the vector. Once the constructs were cloned, multiple clones of the open reading frame of the chimeric LC and HC were sequenced. The sequences of the chimeric LC and HC were compared to those of mAb6B5 and huC_{K} and huC_{G2} to confirm that they encoded the correct chimeric sequence. The nucleotide sequences and the deduced amino acid sequence of the ch-mAb6B5 LC and HC are presented in FIG. 10 (SEQ ID Nos. 15, 16) and 11A and 11B (SEQ ID Nos. 17, 18) respectively.

Expression of Chimeric mAb6B5

The next goal was to establish a stable cell line expressing ch-mAb6B5. The ch-mAb6B5 HC and LC constructs (produced from McLean's vectors) were linearized with Pvul and co-transfected into the P3X non-producing murine myeloma according to the manufacturer's instructions). Following transfection, the cells were diluted in growth medium and plated into 96-well tissue culture plates at 8×10³ cells per well in 200 μl media. The cells were re-fed after 24 hours with media containing 3.0 μg/ml puromycin dihydrochloride (Sigma). After 13 days, supernatants from growing colonies were screened by a sandwich ELISA for the presence of chimeric antibodies.

For identifying cells producing the anti-PCP chimeric mAb6B5 a “sandwich” ELISA was used to screen colonies growing in the 96 well plates. The screening sandwich ELISA was designed to detect assembled (consisting of heavy and light chains) human IgG-kappa antibodies. The antibody used to coat the plates was a goat anti-human IgG antibody and the detection antibody was a goat anti-human kappa antibody.

Briefly, flat-bottomed 96-well plates were coated overnight at 4° C. or for 2 hr at 37° C. with 100 μl of 5 μg/ml goat anti-human IgG (γchain specific) (Southern Biotechnology Associates, Inc., Birmingham, Ala.). Plates were washed with PBS; blocked with 100 μl of 3% bovine serum albumin (BSA) in PBS overnight at 4° C.; and washed again. Then 50 μl of transfected cell supernatant was added to each well. Following overnight incubation at 4° C. or for 2 hr at room temperature, plates were washed, and 100 μl alkaline phosphatase-labeled goat anti-human kappa chain (Southern Biotechnology Associates, Inc.) diluted 1:1000 with 1% BSA in PBS was added to each well for 1 hr at 37° C. After washing extremely well, 100 μl substrate (p-nitrophenyl phosphate) was added to the wells. The plates were incubated at 37° C. After 15 mins, the OD₄₀₅ readings were determined every 30 minutes on an ELISA plate reader. All positive clones were retested to confirm positivity. Three positive clones were identified and retested twice.

To confirm that the antibodies produced by the clones are anti-PCP antibodies, the supernatants were tested for PCP-binding using an ELISA format. Briefly, 96-well plates were coated with the PCP like hapten PCHAP conjugated with ova albumin (PCHAP-ova) diluted in coating buffer (0.1M carbonate buffer, pH 9.6) at 100 ng/well, then washed and blocked overnight at 4° C. with Super Block (Pierce, Rockford, Ill.). Supernatents (50 μl) from transfected cells were added to each well and incubated overnight at 4° C. or 2 hr at room temperature. After washing, 100 μl of alkaline phosphatase-labeled goat anti-human IgG (γ specific) diluted 1:1000 was added to each well and incubated for 1 hr at 37° C. After washing, substrate was added to the wells and the OD₄₀₅ readings were determined as described above. The ELISA was repeated on the same supernatants with an alkaline phosphatase labeled-goat anti-mouse (Fc specific) antibody to confirm that the HC of the PCP binding antibodies expressed from the transfected cells were human, not mouse. The antibodies from both clones were positive for PCHAP-binding (a PCP-like hapten). One of the clones produced extremely small amounts of antibody, as determined by a quantitative sandwich ELISA, thus it was not tested further. The clone that was left was named ch-mAb6B5 for chimeric anti-PCP mAb6B5.

To purify and concentrate ch-mAb6B5 for further testing, a protein-G column and HPLC were used. After binding to the column, ch-mAb6B5 was eluted with glycine-Hcl at pH 2.5. The purified protein was then dialyzed again in a physiological phosphate buffer.

To confirm that the PCP-binding antibody produced by clone ch-mAb6B5 was chimeric and had the human kappa and IgG constant regions, an immunoslot blot procedure was performed. Briefly, equal concentrations of purified ch-mAb6B5 or native (murine) mAb6B5 resuspended in phosphate buffered saline (PBS) were applied directly to a standard nitrocellulose membrane using a Millipore dot slot apparatus (Millipore). After blocking overnight with 3% BSA in PBS, the blots were incubated with one of the following alkaline-phosphatase labeled antibodies: goat anti-human IgG (Fc specific) (Caltag, Burlingame, Calif.), murine mAb anti-human kappa (Southern Biotechnology) or goat anti-mouse (Fc specific) (Sigma). After washing, the blot color was developed using a Alkaline Phosphatase Conjugate Substrate kit (Bio-Rad, Hercules, Calif.).

As shown in FIG. 12, native mAb6B5 was detected only with the anti-mouse (Fc specific), while ch-mAb6B5 was detected with anti-human kappa and anti-human IgG (Fc specific). There was a slight cross-reaction with anti-mouse IgG (Fc specific). The data from ELISA and immunodot slot indicated that a stable cell line expressing an anti-PCP antibody that is human kappa positive was produced. Additionally, it also indicated that the anti-PCP antibody (ch-mAb6B5) had human IgG Fc and not mouse Fc regions. These data confirmed that the chimeric form of mAb6B5 (ch-mAb6B5) was produced.

Characterization of ch-mAb6B5

For ch-mAb6B5 to function in humans as mAb6B5 functions in rats, it is imperative that it retains the same characteristics, especially affinity and specificity. To determine if genetic manipulation altered the affinity of the PCP binding site, the IC₅₀ of ch-mAb6B5 was determined by two methods-radioimmunoassay (RIA) and competitive ligand ELISA.

Briefly, a RIA that was used to determine affinity of ch-mAb6B5 for PCP could be explained as follows. A 100 μl aliquot of [³H]PCP (30,000-40,000 dpm) in RIA buffer (50 mM Tris-HCl adjusted to pH 7.6, 0.15M NaCl, 0.1% BSA and 0.2% NaN₃) was added to 50×14 mm sample tubes (Sarsedt, Princeton, N.J.). Then 100 γl of appropriate concentration of test ligand was added to duplicate tubes. The ch-mAb6B5 antibody was diluted in RIA buffer to a concentration, which would bind 15 to 20% of the [³H]PCP in the absence of PCP. A 100 μl aliquot of this dilution was then added to all tubes except the tubes for non-specific binding. To nonspecific binding tubes 100 μl of RIA buffer was added. After vortex mixing, the tubes were incubated at 4-8° C. overnight. Next, 1 ml of RIA buffer containing 5% goat anti-mouse IgG (Pel-Freez Biologicals, Rogers, Ark.) and 5% polyethylene glycol 8000 (J. T. Baker Chemical Co.) was added. The tubes were incubated for 15 min at 4-8° C. and centrifuged at the same temperature for 15 min at 2000×g to precipitate the antibody-bound reactivity. The supernatant fluid was aspirated and the pellet was resuspended in the same test tube with 2 ml of scintillation fluid (Liquiscint, National Diagnostics, Manville, N.J.). The tube was placed in a 7 ml scintillation vial and the concentration of [³H]PCP was determined by liquid scintillation spectrometry.

The competitive ligand ELISA that was used to determine the IC₅₀ of ch-mAb6B5 for PCP and other arylcyclohexylamines such as TCP and PCE is explained as follows. Briefly, a PCP-binding ELISA was performed as described earlier with the following exception. PCP, TCP or PCE was diluted to various concentrations (representing a full dose-response curve) and added to the wells simultaneously with the supernatant of the ch-mAb6B5 producing cell lines.

In the RIA, the IC₅₀ is defined as the concentration of cold antigen at which the antibody binding of the radiolabeled antigen is decreased by 50%. In Inhibition ELISA, the IC₅₀ is the concentration of liquid phase antigen at which antibody binding of the plate-bound antigen is decreased by 50%. The IC₅₀ for both methods very closely approximates the affinity of an antibody. Native mAb6B5, as previously determined by the RIA, has an IC₅₀ of 1.3 nM. The IC₅₀ of ch-mAb6B5 as determined by RIA was 1.6 nM. The test results of the inhibition ELISA were similar (FIG. 13). The IC₅₀ of mAb6B5=2.3 nM and the IC₅₀ of ch-mAb6B5=3.0 nM. Thus testing by both techniques demonstrated that the IC₅₀, thus the affinity, of ch-mAb6B5 for PCP is the same as native mAb6B5.

Native mAb6B5 has the capability of cross-reacting with other potent arylcyclohexylamines such as TCP and PCE. This is an important characteristic in that mAb6B5 could be a medication for treating abuse of a whole class of drugs. To determine if ch-mAb6B5 retains this unique cross-reactive property, the IC₅₀ of ch-mAb6B5 for TCP, PCE and PCP was determined with an Inhibition ELISA (FIG. 14). The IC₅₀ of ch-mAb6B5 for PCP=3.0 nM, for PCE=4.8 nM and for TCP=18.0 nM. In that study, by RIA, mAb6B5 bound to PCE as strongly as PCP and TCP 2 times less than PCP. The data in this study demonstrated that ch-mAb6B5 had retained its specificity for other structurally similar arylcyclohexylamines, which are also dangerous drugs of abuse.

The chemical characteristics of native mAb6B5 and ch-mAb6B5 were compared. As shown in Table 3, the two antibodies were almost identical in their size, number of amino acids and isoelectric point. These properties are important in that they can affect the function of an antibody in vivo. At least in regard to size and electric charge, the two antibodies were functionally the same.

TABLE 3
Chemical properties of anti-PCP mAb6B5 and ch-mAb6B5
light chain (LC) and heavy chain (HC)

| | | Molecular Weight | |
|---|---|---|---|
| | # Amino Acids | (kD) | Isoelectric point^{a} |
| mAb6B5 LC | 219 | 24157 | 6.48 |
| ch-mAb6b5 LC | 218 | 23804 | 6.48 |
| mAb6b5 HC | 441 | 48813 | 7.27 |
| ch-mAb6B5 HC | 443 | 48717 | 7.24 |

### EXAMPLE 6

Large Scale Production of Chimeric mAb6B5 in Mammalian Cells

The genes for the chimeric mAb6B5 will be stably expressed in dihydrofolate reductase-negative (dhfr−) Chinese hamster ovary cells (CHO) using the dhfr amplification system and a murine myeloma cell line. Clones producing the highest concentrations of functional c-mAb6B5 are identified and adapted to anchorage-independent growth. These clones are grown in bioreactors for large-scale production of chimeric mAb6B5 to be used in preclinical experiments described below.

The CHO/dhfr− mammalian expression system has been widely used in industry and academic research to produce large quantities of recombinant proteins (Geisse and Kocher, 1999). The CHO cell expression system has many advantages. First, the transgenes are integrated into the genome of the cells, producing stable cell lines. Second, the cells are easy to transfect and have good growth characteristics in serum-free and serum-containing media. Third the system has been extensively characterized. Finally, CHO cells are highly suitable for the induction of gene amplification mechanisms, which can greatly increase recombinant protein production. The dhfr is a commonly used amplification marker (Geisse and Kocher, 1999).

For the transfection of the chimeric mAb6B5 genes into CHO cells, plasmids pSV2-dhfr (ATCC, #37146), pHC-huCγ₂, and pLC-huCₖ (containing the genes for dhfr, chimeric mAb6B5 heavy chain and chimeric mAb6B5 light chain, respectively) are linearized with the appropriate restriction enzyme and introduced into CHO cells together by DNA-liposome-mediated transfection using Lipofectamine 2000 (Invitrogen) according to manufacturers protocol. The transfected cells are incubated at 37° C. in fresh media for 48 hr before they are harvested. They are then resuspended in media containing the selection drugs, histidinol and mycophenolic acid, and plated in 96-well plates. Approximately twelve days after adding selection media the supernatants from the growing clones are screened by enzyme-linked immunoabsorbent assay (ELISA) to test for secretion of heavy and light chains. Immunolon II 96 well plates are coated with goat anti-human-IgG in carbonate buffer at pH 9.6, and blocked with 3% BSA. Supernatants from the tranfectants are added and the plates are incubated overnight at 4° C. After washing, plates are developed with goat anti-human-kappa conjugated with alkaline phosphatase to detect cell lines that are secreting heavy and light chain.

High producing clones are expanded and replated in 96-well plates in the presence of 5 nM MTX (Sigma) to induce amplification of dhfr and the chimeric mAb6B5 genes. ELISA is used to test clones for antibody production and the highest producing clones are selected. The process is sequentially repeated with 50 nM, followed by 250 n M MTX. The highest producing clones are expanded and adapted for anchorage-independent growth by using media and protocol published by Sinacore et al (2000). Finally, high producing clones that can grow in an anchorage dependent manner are expanded for antibody production in bioreactors. These cells are continually cultured in 250 nM MTX to maintain selection. It is important to note that CHO cells are adherent cell lines and are cultured by protocols for such until they are adapted to anchorage-independent growth.

Using CHO cells to produce antibodies is a popular and well-characterized technique. However, the development of CHO cell clones will be labor intensive due to the amplification and anchorage-independent adaptation procedures. Alternatively, ch-mAb6B5 can be produced myeloma cell lines.

The antibodies from high producing clones of CHO cells or myeloma cell lines are analyzed for assembly and functional capacity. SDS-PAGE and immunoblotting are used to characterize the qualitative and quantitative aspects of the antibody. The PCP binding affinity (K_{D}) of c-mAb6B5 is then determined using [³H] PCP and equilibrium dialysis (McClurkan et al., 1993). To determine specificity for arylcyclohexylamines, the chimera can be tested in a RIA format using [³H] PCP as the radioligand and a series of arylcyclohexylamines.

For large-scale antibody production and purification, Biostat® B autoclavable bench top bioreactor (B. Braun Biotech Inc., Allentown, Pa.) can be used to generate from 1-10 g of antibody every two weeks. Purification of the antibody will be carried out as follows. After production, the antibody-containing bioreactor culture media is centrifuged and diluted 1:5 with deionized water. The pH of the diluted mixture is adjusted to 6.0 with concentrated HCl. The sample is loaded on a large chromatography column packed with SP-Sepharose Big Bead media (Pharmacia LKB Biotechnology) and washed with 50 mM MES buffer (pH 6.0) to remove non-specifically bound proteins. The IgG is then eluted in one step using 50 mM MES/0.15 M NaCl. This elution also serves to concentrate the antibody. The purified antibody is concentrated and the buffer exchanged to sterile PBS using an ultra filtration device. Aliquoted samples are stored in the −80° C. freezer. Immediately before injection into an animal, the antibody is quickly thawed at 37° C. (to prevent formation of immune complexes), ultracentrifuged at 100,000×g for 1 hr followed by low speed centrifugation at 3800 rpm for 15 min to remove possible aggregate formations. The protein concentration is determined by spectrophotometer. As part of the purification process, the purity and functionality of the antibody are determined by SDS-PAGE and binding assays. The antibody preparation will also be checked for the content of endotoxins.

### EXAMPLE 7

Effectiveness and Safety of Chimeric mAb6B5 in Reversing In Vivo Effects of PCP Drug Abuse

Pharmacokinetic and behavioral studies in rats are performed to compare the chimeric mAb6B5 to native murine mAb6B5 for its ability to reduce adverse effects induced by PCP and other arylcyclohexylamines, such as TCP and PCE. These data predict the potential efficacy of the chimeric mAb6B5 in phase I clinical trials. Additionally, the immunogenicity of the chimeric mAb6B5 and native mAb6B5 is compared in rats.

Serum Concentration of Chimeric Antibody

To compare the half-lives of native mAb6B5 and chimeric mAb6B5, native or chimeric antibodies are administered to rats i.v. and serum concentrations of the antibodies will be measured over time. The reported t_{1/2λz} (the terminal elimination half life) of murine monoclonal antibodies is 5-8 days in rats. However, studies with native mAb6B5 suggest a functional t_{1/2λz} (determined by measuring long term binding of PCP in the serum) of about 15 days (Proksch et al., 2000). The biological t_{1/2λz} appears to underestimate the functional t_{1/2λz} for protective effects. To sort out these seemingly conflicting findings, serum concentrations of chimeric mAb6B5 are measured for 4-7 half lives, and until the pharmacological effects have ceased (as determined by behavioral experiments). PCP protein binding in serum will also be measured as part of the pharmacokinetic studies. Direct comparison of biological and functional t₁₂ for both antibodies will help to sort out differences and similarities in antibodies.

Analysis of serum concentration of chimeric and native mAb6B5 will be performed using an antibody capture ELISA. For the assay, ELISA plates are coated with a drug hapten-protein conjugate. Serum samples containing the antibody will be added to the wells and bound antibody will be detected with an anti-human IgG peroxidase conjugate (for chimeric mAb6B5) or an anti-mouse IgG peroxidase conjugate (for native mAb6B5). Following incubation with the appropriate substrate, the absorbance at 405 nm is read on a microtiter plate reader. The amount of IgG in the sample is determined by comparison to a serum standard curve from the appropriately matched IgG.

Pharmacokinetic and Behavioral Studies in Rats

The pharmacokinetics of chimeric mAb6B5:PCP interactions are examined and compared to the interactions of native mAb6B5: PCP using two general animal models of human drug abuse patterns, acute PCP overdose and chronic PCP use. To simulate a model of substantial human overdose, rats are administered a one time, intravenous dose of 3 mg/kg PCP (Hardin et al., 1998). To simulate a pattern of chronic use of PCP, the rats are chronically infused with a high dose (18 mg/kg/day) of PCP via a subcutaneous osmotic pump as described previously. In the acute overdose model, native or chimeric mAb6B5 is administered shortly after PCP is given. In the chronic use model, the native or chimeric antibody is administered when PCP serum levels have reached steady-state (24 hours after the minipumps have been implanted). In both models, PCP concentrations are measured at various points in serum, brain and testis. In addition, antibody concentrations are measured at various time points in serum.

In a series of behavior studies, the efficacy and duration of action of chimeric mAb6B5 and native mAb6B5 in reversing the adverse effects of PCP, PCE and TCP are compared. The effectiveness of the antibody is gauged by its ability to block drug-induced locomotor activity and stress. These experiments will use the same design, controls, and doses of drug and antibody as used in previous studies of native mAb6B5 (Hardin et al., 1998 and 2002; Proksch et al., 2000). Both forms of mAb6B5 are tested in the acute overdose model and the chronic use model.

For all animal experiments male Sprague-Dawley rats with dual cannula implanted in the right jugular and femoral veins will be purchased from Hilltop lab Animals (Scottsdale, Pa.). Blood samples are obtained via the femoral cannula; and drug and antibody is administered via the jugular vein cannula. In experiments simulating chronic use, PCP is continuously infused via a subcutaneous osmotic minipump. The pumps are implanted between the scapulae of the rats under halothane anesthesia as previously described. To monitor locomotor activity for the behavioral experiments, the rats will be placed in open-top polyethylene chambers (60×45×40 cm) and spontaneous behavior recorded by a video camera located above the chambers. The video camera is connected to an S-VHS recorder and monitor. Analysis of the behavior videotapes is carried out with Ethovision software (Noldus Information Technology, Inc., Sterling, Va.). This software quantifies distance traveled by the rats during the behavioral session. All animal experiments will consist of 6-8 animals per treatment group. Previous experiments in this lab using similar sample sized produce results with variances of less than 15%.

Pharmacokinetic and pharmacodynamic analyses are carried out using the WinNonlin pharmacokinetic software package (Pharsight Corp., Cary, N.C.). Model-dependent pharmacokinetic analysis of PCP, chimeric mAb6B5 and mAb6B5 concentration-time data is performed using a nonlinear least-squares curve fitting routine (WinNonlin, Pharsight, Inc, Mountain View, Calif.).

Immunogenicity of Chimeric mAb6B5

To compare the potential immunogenicity of the chimeric mAb6B5 and native mAb6B5, both antibodies are administered to groups of rats multiple times over a period of 6-8 weeks. Serum samples are collected at regular intervals during the dosing period and tested for the presence of anti-murine IgG antibodies (in the case of native mAb6B5), anti-human IgG (in the case of ch-mAb6B5), or anti-idiotypic antibodies (for both forms). The general health status of the animals is also followed during the experiments. At the end of the antibody-dosing period, an experiment will be performed to determine if a neutralizing immune response has been mounted as a result of the long-term administration of chimeric or murine antibody. The rats are given a single dose of PCP and antibody (either native or chimeric, depending on the form previously administered) and their PCP-induced locomotor activity measured. It is anticipated that ch-mAb6B5 will be more antigenic in rats than murine mAb6B5. However, ch-mAb6B5 will have low to no significant antigencity when used in humans because of its human immunoglobulin constant regions.

Analysis of an immune response against chimeric and native mAb6B5 is also performed with an ELISA capture assay. To measure a total immune response, ELISA plates are coated with either native mAb6B5 or chimeric mAb6B5, and the detecting antibody will be goat anti-rat IgG peroxidase conjugate. To measure an anti-idiotypic response, the wells will be coated with the Fab of chimeric mAb6B5 (which is the same structure on native mAb6B5), and the detecting antibody will be goat anti-rat peroxidase conjugate. Analysis of PCP concentrations in the serum and brain will be performed by solid phase extraction followed by radioimmunoassay as previously described (Proksch et al., 2000). PCP protein binding in serum samples will be determined by equilibrium dialysis as previously described (Proksch et al., 2000).

The following references were cited herein:
Clark, (2000) Immunol Today 21:397-402.
Coloma et al., (1992) J Immunol Methods 152:89-104.
Geisse and Kocher, (1999) Methods Enzymol 306:19-42.
Hardin et al., (1998) J Pharmacol. Exp. Ther. 285:1113-1122.
Hardin et al., (2002) J Pharmacol. Exp. Ther. 302:119-126.
Kunert et al., (2000) Biotechnol. Bioeng. 67:97-103.
Kuus-Reichel et al., (1994) Clin. Diagn. Lab Immunol. 1:365-372.
Laurenzana et al., (2003) J Pharmacol. Exp. Ther. 306:1092-1098.
Lim et al., (1998) J. Biol. Chem. 273:28576-28582.
McClurkan et al., (1993) J Pharmacol. Exp. Ther. 266:1439-1445.
McLean et al., (2000) Mol. Immunol. 37:837-845.
Morrison, (1992) Annu. Rev. Immunol. 10:239-265.
Morrison, (1994) Cloning, Expression, and Modification of Antibody V Regions, in Current Protocols In Immunology, p. 2.12.11-12.12.17.
Owens et al., (1988) J Pharmacol. Exp. Ther. 246:472-478.
Page and Sydenham, (1991) Biotechnology (N Y) 9:64-68.
Peakman et al., (1994) Hum Antibodies Hybridomas 5:65-74.
Proksch et al., (2000) J Pharmacol. Exp. Ther. 292:831-837.
Reichert, (2001) Nat. Biotechnol. 19:819-822.
Sinacore et al., (2000) Mol. Biotechnol. 15:249-257.
Valentine and Owens., (1996) J. Pharmacol. Exp. Ther. 278:717-724.
Valentine et al., (1994) J Pharmacol. Exp. Ther. 269:1079-1085

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of treating arylcyclohexylamine drug abuse, the method comprising administering a chimeric mouse/human monoclonal antibody specific for phencyclidine or phencyclidine-like drugs to a subject, wherein administration of the chimeric antibody modulates the adverse effects of arylcyclohexylamine drug abuse.

2. The method of claim 1, wherein the arylcyclohexylamine is selected from the group consisting of phencyclidine (PCP), 1-[1-(2-thienyl)cyclohexyl] piperidine (TCP), and N-ethyl-1-phenylcyclohexylamine (PCE) or other structurally similar, psychoactive analogs thereof.

3. The method of claim 1, wherein the mouse/human chimeric antibody comprises the variable regions of mAb6B5.

4. The method of claim 1, wherein the subject is selected from the group consisting of a rodent, a non-human primate, and a human.

5. The method of claim 4, wherein the subject is using phencyclidine.

6. The method of claim 4, wherein the subject overdosed on phencyclidine.

7. The method of claim 1, wherein the chimeric antibody decreases the concentration of phencyclidine (PCP), 1-[1-(2-thienyl)cyclohexyl] piperidine (TCP), or N-ethyl-1-phenylcyclohexylamine (PCE) in the brain of the subject.

8. The method of claim 1, wherein the pharmaceutically effective amount is about 5 mg to about 45 mg of chimeric antibody per kilogram of subject body weight.

9. A method of treating phencyclidine drug abuse, the method comprising administering a pharmaceutically effective amount of a chimeric mouse/human monoclonal antibody specific for phencyclidine to a subject, wherein administration of the chimeric antibody modulates the adverse effects of phencyclidine drug abuse.

10. The method of claim 9, wherein the mouse/human chimeric antibody comprises the variable regions of mAb6B5.

11. The method of claim 9, wherein the subject is selected from the group consisting of a rodent, a non-human primate, and a human.

12. The method of claim 11, wherein the subject is using phencyclidine.

13. The method of claim 11, wherein the subject overdosed on phencyclidine.

14. The method of claim 9, wherein the chimeric antibody decreases the concentration of phencyclidine in the brain of the subject.

15. The method of claim 9, wherein the pharmaceutically effective amount is about 5 mg to about 45 mg of chimeric antibody per kilogram of subject body weight.

16. A method of decreasing the concentration of phencyclidine in the brain of a subject, the method comprising administering a pharmaceutically effective amount of a chimeric mouse/human monoclonal antibody specific for phencyclidine to the subject.

17. The method of claim 16, wherein the subject is selected from the group consisting of a rodent, a non-human primate, and a human.

18. The method of claim 17, wherein the subject is using phencyclidine or overdosed on phencyclidine.

19. The method of claim 16, wherein the mouse/human chimeric antibody comprises the variable regions of mAb6B5.

20. The method of claim 16, wherein the pharmaceutically effective amount is about 5 mg to about 45 mg of chimeric antibody per kilogram of subject body weight.
